(19) United States
(12) Patent Application Publication
(10) Pub. No.: US 2007/0270677 A1
(43) Pub. Date: Nov. 22, 2007

(54) Interactive device for monitoring and reporting glucose levels

(76) Inventor: Steve Thuss, Wellington, FL (US)

Correspondence Address: Mitchell A. Stein, Esq.;STEIN LAW, P.C., Suite 4, 24 Woodbine Avenue, Northport, NY, 11768, US

(21) Appl. No.: 11/438,566

(22) Filed: May 22, 2006

(51) Int. Cl. A61B 5/00

(52) U.S. Cl. 600/365

(57) ABSTRACT

A device for determining glucose levels in a patient having a CPU for receipt and analysis of data; a glucose tester means for testing the amount of glucose in the patient at the point of the test, providing the same information to the CPU, and the CPU for determining the amount; a database for storing glucose data linked to the patient; a display for displaying glucose levels and interfacing with the patient; and a voice processor for processing the glucose level and synthesizing an auditory output that conforms with the glucose level and optionally includes further instructions as determined by a physician and the CPU after analysis of the amount of glucose. The device further provides for data acquisition via the voice processing and/or keyboard for acquiring the following data concurrently with the glucose level then tested: (1) dietary consumption; (2) exercise; (3) medical information (size, BMI, other conditions, other medications, etc.); (4) comments and instructions. The digital storage links the information per patient for later data retrieval. The device has an input/output to the physician. The physician's instructions are provided and output via the voice processor and/or a visual display.

### FIELD OF THE INVENTION

The present invention relates to the field of diabetes management and more particularly to glucose meters for the self-monitoring of blood glucose wherein patient-relevant information associated with the disease state is flagged and stored with the patient's active data such that the patient and physician can have immediate access and render modifications and monitoring as necessary.

### BACKGROUND OF THE INVENTION

The instant invention relates to a glucose meter which incorporates a means for the patient to record time-specific data relevant to diabetes management in order to either self-evaluate or have a physician evaluate the correlation between inputted data and the patient's recorded blood glucose level.

Diabetes mellitus, commonly referred to as diabetes, is a chronic disease in which an individual's blood glucose levels become abnormally high due to an inability to break down glucose. The hormone insulin is responsible for regulating glucose levels in the blood. Diabetics produce either a deficient amount of insulin to break down the glucose present in the blood, or are resistant to insulin and therefore cannot use it properly. An estimated 17 million persons in the United States have diabetes, with almost 1 million new cases being diagnosed each year.

Diabetes is known to cause damage to the small and large blood vessels, leads to diabetic blindness, kidney disease, amputations of limbs, stroke, and heart disease. According to the U.S. Centers for Disease Control and Prevention, more than 3 million Americans who have diabetes are visually impaired.

Self-monitoring of one's blood glucose has been determined to be an effective tool to manage diabetes. Self-monitoring of blood glucose is recommended by the FDA for all people with diabetes. It is recognized that self-monitoring of blood glucose will allow the user to: (1) keep track of their glucose levels over time, (2) make day-to-day decisions for managing glucose, (3) recognize emergency situations; and (4) educate themselves on how to manage their blood glucose levels. An estimated 8% of the 17 million American currently suffering from diabetes monitor their blood glucose at home.

Checking one's blood glucose level allows a physician and/or the individual to determine how much insulin should be taken to maintain normal blood glucose levels. The amount of glucose a diabetic requires will likely vary with age, a change in diet or lifestyle, stress, or illness. It is therefore necessary for a diabetic to periodically check his or her glucose levels to ensure they are taking the proper amount of insulin and to render changes to diet, exercise and the like to improve management and longevity.

Many diabetes management plans direct the individual to test glucose three or more times a day under normal conditions, and more frequently during times of illness or stress. Typically diabetics are directed to check their blood glucose levels before meals, after meals, at bedtime, and any other time they experience signs or symptoms of hyperglycemia or hypoglycemia. However, the specific times and frequency a diabetic should conduct such checks are typically determined by the individual's physician or by the happening of an event, rather than correlating to normal life styles, like ingestion times, quantities and types of food, exercise, stress, illness and the like.

Many factors can cause one's blood glucose levels to fluctuate such as the amount of insulin taken, the amount of food ingested (or not ingested), the type of food ingested, and the amount one exercises. As such it is preferable for a diabetic to keep records of not only periodic blood glucose levels, but information regarding diet and exercise and to report this information to the treating physician to determine proper medical intervention.

Glucose meters measure the amount of glucose present in an individual's blood. To use a glucose meter, the user typically places a small sample of blood on a test strip. A chemical present on the test strip (typically glucose oxidase, dehydrogenase, or hexokinase) then combines with the blood to create a reaction. When the test strip is inserted in to a glucose meter, the meter measures the chemical reaction and translates it into a score indicating the individual's blood glucose level. The score is often displayed or printed. Glucose meters have also been developed which measure the presence of glucose by measuring the amount of electricity that can pass through a sample of blood, or how much light reflects from the sample, but these are complex and generally less reliable in testing actual blood glucose levels.

Among the problems associated with the self-monitoring of blood glucose levels are the ability to associate a given score from a glucose meter with the diet and activities (and remainder of an individual's regimen). It has heretofore been left to the individual to manually record data such as when they ate, what they ate, when they exercised, how long they exercised, and the like typically after an event by memory to the physician to understand the correlation with such activity and the individual's blood glucose levels. Virtually heretofore unknown was the ability, even manually, or desire to maintain such records in the absence of a triggering event that would require someone to do perform the same. The instant device provides a simple ability for patient compliance and the rendering of data virtually simultaneously with daily activities in a simple manner. It is recognized that an understanding of said correlation will allow the physician and/or patient to accurately determine the proper amount of insulin needed, or whether a simple change in lifestyle would suffice to bring the individual's blood glucose level within a normal range. Thus, it is an object of the instant invention to provide a glucose meter which allows the user to record information relating to their diet, amount of exercise, level of stress or illness and other circumstances concurrently with the user's periodic testing and recording of their blood glucose level.

Known in the art are glucose meters which include audio output to aid a user with vision loss in self-monitoring blood glucose levels. One such device is commercially available from Roche Diagnostics under the trade name “Accu-Chek Voicemate.” This single unit device provides both audio instruction and audible test results. Also commercially available are speech synthesizers which attach to glucose meters to similarly provide both audio instruction and audible test results. Examples of such commercially available voice synthesizers are the “Voice-Touch” speech synthesizers produced by Myna Corporation for use with LifeScan glucose meters, and “Digi-Voice” speech synthesizers produced by Science Products also for use with certain LifeScan glucose meters. Also known in the art are glucose meters which aid the user in choosing the correct insulin containers for insulin injection. For example, U.S. Pat. No. 5,786,584 to Button et. al. discloses a vial and cartridge reading device for reading the labeled contents of an insulin container and then providing an audible message informing the user of the labeled contents.

Heretofore unknown are glucose meters which incorporate a recording means and speech synthesizer for audio output of recorded information (or virtually concurrent recordings, stored for later viewing). Thus, it is a further object of the present invention to provide a glucose meter which incorporates a recording means and voice synthesizer in one device to speak instructions, test results, and recorded information to the user aloud as well as to display the same.

Further objects will become apparent to one of ordinary skill in the art upon the understanding of the instant invention.

### SUMMARY OF THE INVENTION

A device for determining glucose levels in a patient having a CPU for receipt and analysis of data; a glucose tester means for testing the amount of glucose in the patient at the point of the test, providing the same information to the CPU, and the CPU for determining the amount; a database for storing glucose data linked to the patient; a visual display for displaying glucose levels and interfacing with the patient; and a voice processor for processing the glucose level and synthesizing an auditory output that conforms with the glucose level and optionally includes further instructions as determined by a physician and the CPU after analysis of the amount of glucose. The device further including data acquisition via the voice processing and/or keyboard for acquiring the following data concurrently with the glucose level then tested: (1) dietary consumption; (2) exercise; (3) medical information (size, BMI, other conditions, other medications, etc.); (4) comments and instructions. The digital storage links the information per patient for later data retrieval.

In accordance with the teachings of the instant invention, an interactive glucose meter is disclosed which includes an input/output to the physician, by which the physician can evaluate the recorded blood glucose results and other information inputted by the patient for analysis, thereby eliminating the need for the patient to schedule an appointment to meet with the physician in person. The physician's instructions are provided and output via the voice processor and/or the visual display.

In accordance with the teachings of the present invention, a glucose meter is provided which allows the user to record information relating to their diet, amount of exercise, level of stress or illness and other circumstances concurrently with the user's periodic testing and recording of their blood glucose level so as to flag and store the data with the patient's active data such that the patient and physician can have immediate access and render modifications and monitoring as necessary. Said flagging will allow the physician or individual to better understand the correlation between the patient's lifestyle and his or her blood glucose level. The user may input the desired information via a keyboard which may be incorporated with the glucose meter or attached to the glucose meter's data port. Alternatively, information may be recorded via a recording device incorporated directly into the glucose meter or attached to the glucose meter's data port. The voice recordings can be recorded in *.wav or other format.

In accordance with the teachings of the present invention, a glucose meter is provided which incorporates a voice processor/synthesizer to both record spoken data and speak instructions and results to the user aloud. The synthesizer's voice can be either male or female, and may be translated into a multiplicity of languages without deviating from the spirit of the instant invention. The volume of the voice synthesizer may increased or reduced depending on the user's preference. The voice synthesizer may alternatively be reduced to zero, should the user desire silence, in which case the instructions and/or results may be viewed directly on the display screen.

In accordance with the teachings of the present invention, a glucose meter is provided which incorporates a large display screen to aid the visually impaired or for people who are assisting them. The display screen functions independently of the voice processor/synthesizer so as to allow the user to have the instructions and/or results presented via the display screen and/or the voice synthesizer.

The various features of novelty which characterize the present invention are expressly and unambiguously delineated in the claims annexed to and forming part of the disclosure. For a better understanding of the present invention, its practical advantages, and specific objects attained by its use, reference should be had to the drawings and descriptive matter in which there are illustrated and described preferred embodiments of the invention.

Other features will become apparent from reading the disclosure and claims of the instant invention.

### BRIEF DESCRIPTION OF THE DRAWINGS

In the drawings, wherein similar reference characters denote similar elements throughout the several views:

FIG. 1 a topographical overview of the preferred embodiment of the instant invention device;

FIG. 2 is a component diagram of the various hardware components of the preferred embodiment in accordance with the instant invention; and

FIG. 3 is a flow chart of patient entry showing the interface between the various components in accordance with a preferred embodiment of the instant invention.

### DETAILED DESCRIPTION OF THE PREFERRED EMBODIMENTS

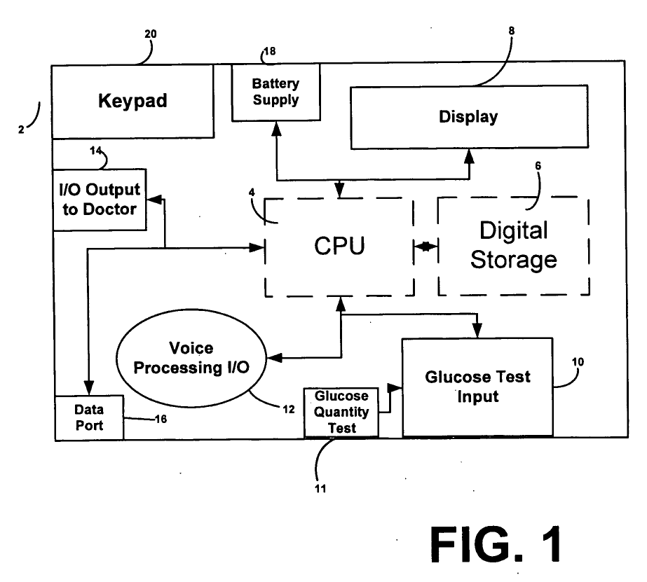

As shown in FIG. 1, at the heart of the device is CPU 4 which engages digital storage 6. It should be recognized that the database, or digital storage 6 is such that each individual user has, associated with a tag specific to the individual one or more of the following items:
(a) glucose levels;
(b) dietary consumption;
(c) exercise;
(d) medical information (size, BMI, other conditions, other medications, etc.); and
(e) comments.

The information indicated to be stored per patient in digital storage 6 and tagged to the individual patient can be maintained for long periods of time, depending upon the size of storage device 6. Likewise, the information can be downloaded from storage device through data port 16 to any other device. It should be appreciated that this information can be hard-wired through port 16, or WI-FI communicated, or any of a number of different mechanisms known or hereinafter developed for the transmission of digital data.

Information can be added to database 6 in a plurality of forms. For example, voice processing I/O allows the patient to enter data by speaking into the monitor 2 and the information is typically digitized and stored (as in a *.wav) file tagged to that patient in digital storage (a/k/a database) 6. Likewise, the patient can enter data via keypad 20. Information is displayed via display 8, or can be spoken back to patient via voice processing I/O 12.

Critical to the device is an “all in one” aspect in which glucose quantities are determined typically by insertion of a glucose strip that has been impregnated with a sample of that patient's blood, and inserted via quantity test, the test is analyzed and output to element 10, and back to CPU 4 for storage in database 6. The glucose reading is tagged to all other information that is stored, as heretofore indicated per patient at specific dates and times such that a physician and/or patient can see all such information or hear all such information stored over a period of time.

I/O output to doctor 14 can occur in the ordinary course of computer science, or can be downloaded via data port 16 and linked to the physician. Also shown is replaceable battery 18, which can be nickel-cadmium (for rechargeability) or lithium (for size) or any of a number of other power supplies. Indeed, the device can be plugged into an AC outlet, provided that a transformer is included to ensure proper power management.

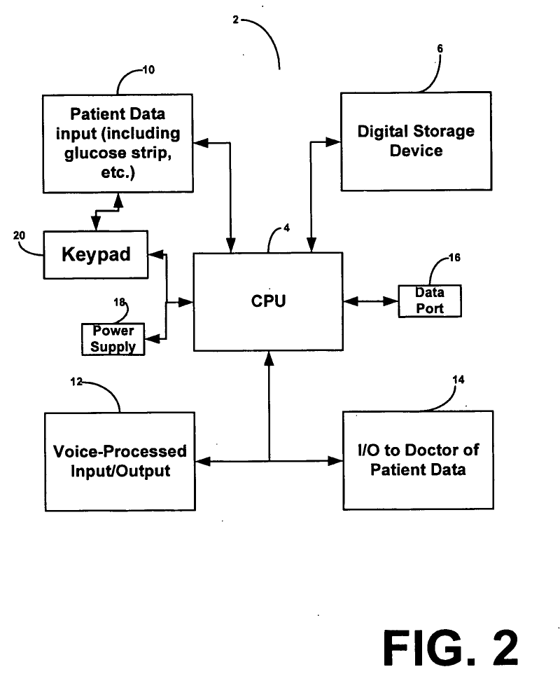

FIG. 2 also shows device 2, in a component blow up model, in which like numbered items have the same function as heretofore indicated.

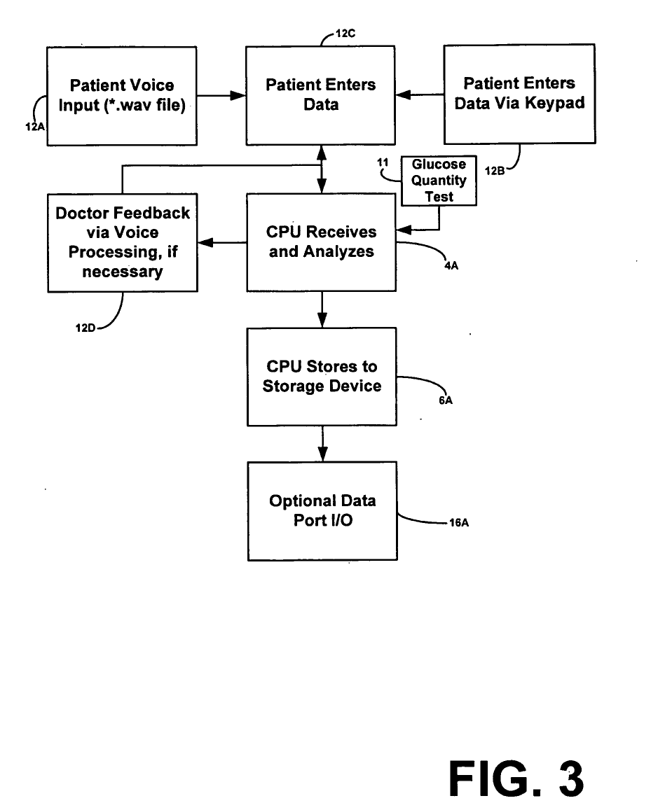

FIG. 3 shows a flow chart wherein patient enters data at step 12C (which can be via keypad 20 if the patient so elects, via step 12B. Also, the patient can engage the voice I/O processor 12 which can not only ask questions and receive answers, but also can simply record the information that the patient provides (including that indicated hereinabove). After (or concurrently) with the input of patient data is glucose quantity test 11 which outputs results to CPU 4A for analysis. Shown also is doctor feedback 12D via voice processing if necessary, or other means (preferably voice interface). When the information is collected it is stored to the storage device via step 6A and output, optionally via data port I/O 16 A.

While there have shown, described and pointed out fundamental novel features of the invention as applied to preferred embodiments thereof, it will be understood that various omissions and substitutions and changes in the form and details of the device illustrated and in its operation may be made by those skilled in the art without departing from the spirit of the invention. It is the invention, therefore, to be limited only as indicated by the scope of the claims appended hereto.

I claim:

1. A device for determining glucose levels in a patient comprising:
(a) a CPU for receipt and analysis of data;
(b) glucose testing means for testing the amount of glucose in the patient at the point of the test, providing the same information to the CPU, and the CPU for determining the amount;
(c) storage means for storing glucose data linked to the patient;
(d) visual display means for displaying glucose levels and interfacing with the patient; and
(e) voice processing means for processing the glucose level and synthesizing an auditory output that conforms with the glucose level and optionally includes further instructions as determined by a treating physician and the CPU after analysis of the amount of glucose.

2. The device of claim 1, further comprising:
(f) data acquisition via said voice processing means and/or said keyboard for acquiring the following data concurrently with the glucose level then tested:
(1) dietary consumption;
(2) exercise;
(3) medical information (size, BMI, other conditions, other medications, etc.);
(4) comments.
(g) wherein said digital storage links the information per patient for later data retrieval.

3. The device of claim 2, wherein said data retrieval includes an input/output to the physician.

4. The device of claim 1, wherein physician's instructions are provided and output via said voice processing means.
